(19) United States
(12) Patent Application Publication
(10) Pub. No.: US 2015/0238549 A1
(43) Pub. Date: Aug. 27, 2015

(54) PROBIOTIC DOSAGE UNITS

(71) Applicant: MAK Wood, Inc., Grafton, WI (US)

(72) Inventors: Mark A. Brudnak, Grafton, WI (US); Eric J. Baer, Grafton, WI (US)

(21) Appl. No.: 14/634,441

(22) Filed: Feb. 27, 2015

Related U.S. Application Data
(60) Provisional application No. 61/945,386, filed on Feb. 27, 2014.

(51) Int. Cl. A61K 35/747; A61K 47/02; A61K 47/24; A61K 35/745; A61K 35/744

(52) U.S. Cl. CPC A61K 35/747; A61K 35/745; A61K 35/744; A61K 47/24; A61K 47/02

(57) ABSTRACT

A dosage unit including a probiotic component, a pH buffering agent, and a tonicity buffering agent. A dosage unit including a probiotic component, a polymeric matrix component, and a tonicity buffering agent, wherein the tonicity buffering agent is present in an amount and configuration suitable to provide a localized isotonic medium upon addition of the dosage unit to 50 mL of 0.1 N HCl (aq).

### CROSS REFERENCE TO RELATED APPLICATIONS

This application claims priority to U.S. Provisional Application No. 61/945,386, filed Feb. 27, 2014, the complete disclosure of which is incorporated by reference in its entirety.

### BACKGROUND

This invention relates to probiotic dosage units.

### SUMMARY

This disclosure provides dosage units comprising a probiotic component, a pH buffering agent, and a tonicity buffering agent.

This disclosure also provides dosage units comprising a probiotic component, a polymeric matrix component, and a tonicity buffering agent, wherein the tonicity buffering agent is present in an amount and configuration suitable to provide a localized isotonic medium upon addition of the dosage unit to 50 mL of 0.1 N HCl (aq).

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “about” is intended to encompass the value that the term is modifying plus or minus an amount that a person having ordinary skill in the art would identify as accounting for user error, instrumental error, or a combination of user and instrumental error.

In one aspect, this disclosure provides a dosage unit. In certain embodiments, the dosage unit may comprise a probiotic component, a pH buffering agent, and a tonicity buffering agent. In certain embodiments, the dosage unit may comprise a probiotic component, a polymeric matrix component, and a tonicity buffering agent.

In certain embodiments, the probiotic component may comprise one or more of a probiotic, a metabolite of a probiotic, an enzyme, and a combination thereof.

In certain embodiments, the probiotic component may be present in an amount from about 50 million to about 500 billion colony forming units (CFU) per dosage unit. In certain embodiments, the probiotic component may be present in an amount of at least about 50 million CFU, at least about 100 million CFU, at least about 150 million CFU, at least about 200 million CFU, at least about 250 million CFU, at least about 300 million CFU, at least about 400 million CFU, at least about 500 million CFU, at least about 600 million CFU, at least about 700 million CFU, at least about 750 million CFU, at least about 800 million CFU, at least about 900 million CFU, at least about 1 billion CFU, at least about 10 billion CFU, at least about 25 billion CFU, at least about 50 billion CFU, at least about 75 billion CFU, at least about 100 billion CFU, at least about 125 billion CFU, at least about 150 billion CFU, at least about 175 billion CFU, at least about 200 billion CFU, at least about 225 billion CFU, at least about 250 billion CFU per dosage unit. In certain embodiments, the probiotic component may be present in an amount of at most about 250 billion CFU, at most about 225 billion CFU, at most about 200 billion CFU, at most about 175 billion CFU, at most about 150 billion CFU, at most about 125 billion CFU, at most about 100 billion CFU, at most about 75 billion CFU, at most about 50 billion CFU, at most about 25 billion CFU, at most about 10 billion CFU, at most about 1 billion CFU, at most about 900 million CFU, at most about 800 million CFU, at most about 700 million CFU, at most about 600 million CFU, at most about 500 million CFU, at most about 400 million CFU, at most about 300 million CFU, at most about 200 million CFU, at most about 100 million CFU, or at most about 50 million CFU per dosage unit.

In certain embodiments, the probiotic component may comprise lactic acid bacteria, yeast, propionic acid bacteria, or a combination thereof.

It should be appreciated that new probiotics are discovered on a regular basis, and that this invention is intended to be applicable to any newly-discovered probiotics that share features in common with the probiotic components disclosed herein. In certain embodiments, the probiotic component may comprise one or more of Lactobacillus acidophilus, Lactobacillus brevis, Lactobacillus casei, Lactobacillus delbrueckii, Lactobacillus gasseri, Lactobacillus fermentum, Lactobacillus helveticus, Lactobacillus paracasei, Lactobacillus plantarum, Lactobacillus reuteri, Lactobacillus rhamnosus , Lactobacillus salivarius , Lactococcus lactis, Bifidobacterium bifidum, Bifidobacterium breve, Bifidobacterium infantis, Bifidobacterium lactis, Bifidobacterium longum, Streptococcus thermophilus, and Saccharomyces boulardii.

Without wishing to be bound by any particular theory, it is believed that spores may possess an innate protection from certain environmental conditions, and therefore, this invention may be intended for use with probiotic components that are not spores. Similarly, without wishing to be bound by any particular theory, it is believed that Bacillales bacteria may possess an innate protection from certain environmental conditions, and therefore, this invention may be intended for use with probiotic components that are not Bacillales bacteria. In certain embodiments, the dosage unit may comprise a spore, a Bacillales bacterium, or a combination thereof in an amount of less than about 0.1% by weight of the dosage unit. In certain embodiments, the dosage unit may comprise a spore, a Bacillales bacterium, or a combination thereof in an amount of less than about 0.1%, less than about 0.05%, less than about 0.01%, less than about 0.005%, or less than about 0.001% by weight of the dosage unit.

In certain embodiments, the tonicity buffering agent may be present in an amount suitable to provide an isotonic medium upon addition of the dosage unit to 50 mL of distilled water. As used herein, an isotonic medium has a water concentration that is approximately equal to the water concentration within a cell or probiotic component. In certain embodiments, an isotonic medium may comprise a water concentration that is at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, or at least about 145% of the water concentration within a cell or probiotic component. In certain embodiments, an isotonic medium may comprise a water concentration that is at most about 150%, at most about 145%, at most about 140%, at most about 135%, at most about 130%, at most about 125%, at most about 120%, at most about 115%, at most about 110%, at most about 105%, at most about 100%, at most about 95%, at most about 90%, at most about 85%, at most about 80%, at most about 75%, at most about 70%, at most about 65%, at most about 60%, or at most about 55% of the water concentration within a cell or probiotic component.

In certain embodiments, the tonicity buffering agent may be present in an amount and configuration suitable to provide a localized isotonic medium upon addition of the dosage unit to 50 mL of 0.1 N HCl (aq). As used herein, a localized isotonic medium has a water concentration in the vicinity of a cell or probiotic component that is approximately equal to the water concentration within the cell or probiotic component.

In principle, a tonicity buffering agent that is capable buffering the tonicity of a medium to enhance the survivability of a probiotic component is suitable for use in this invention. In certain embodiments, the tonicity buffering agent may comprise an inorganic salt. In certain embodiments, the tonicity buffering agent may comprise sodium chloride, potassium chloride, magnesium chloride, calcium chloride, sodium fluoride, potassium fluoride, magnesium fluoride, calcium fluoride, sodium bromide, potassium bromide, magnesium bromide, calcium bromide, sodium iodide, potassium iodide, magnesium iodide, calcium iodide, sodium sulfate, potassium sulfate, magnesium sulfate, calcium sulfate, or a combination thereof.

Under some circumstances, it is desirable to limit the intake of carbonate salts, bicarbonate salts, citrate salts, or lactate salts, so the dosage unit of the present invention may contain little to none of these salts. In certain embodiments, the dosage unit may comprise less than about 0.1% by weight of one or more of a carbonate salt, a bicarbonate salt, a citrate salt, a lactate salt, and combinations thereof. In certain embodiments, the dosage unit may comprise less than about 0.1%, less than about 0.05%, less than about 0.01%, less than about 0.005%, or less than about 0.001% by weight of one or more of a carbonate salt, a bicarbonate salt, a citrate salt, a lactate salt, and combinations thereof.

In certain embodiments, the pH buffering agent may be present in an amount suitable to provide a pH of about 1 to about 7 upon addition of the dosage unit to 50 mL of 0.1 N HCl (aq). In certain embodiments, the pH buffering agent may be present in an amount suitable to provide a pH of at least about 1.0, at least about 1.1, at least about 1.2, at least about 1.3, at least about 1.4, at least about 1.5, at least about 1.6, at least about 1.7, at least about 1.8, at least about 1.9, at least about 2.0, at least about 2.1, at least about 2.2, at least about 2.3, at least about 2.4, at least about 2.5, at least about 2.6, at least about 2.7, at least about 2.8, at least about 2.9, at least about 3.0, at least about 3.2, at least about 3.4, at least about 3.6, at least about 3.8, at least about 4.0, at least about 4.5, at least about 5.0, at least about 5.5, at least about 6.0, or at least about 6.5 upon addition of the dosage unit to 50 mL of the 0.1 N HCl(aq). In certain embodiments, the pH buffering agent may be present in an amount suitable to provide a pH of at most about 7.0, at most about 6.9, at most about 6.8, at most about 6.7, at most about 6.6, at most about 6.5, at most about 6.4, at most about 6.3, at most about 6.2, at most about 6.1, at most about 6.0, at most about 5.9, at most about 5.8, at most about 5.7, at most about 5.6, at most about 5.5, at most about 5.4, at most about 5.3, at most about 5.2, at most about 5.1, at most about 5.0, at most about 4.8, at most about 4.6, at most about 4.4, at most about 4.2, at most about 4.0, at most about 3.5, at most about 3.0, at most about 2.5, at most about 2.0, or at most about 1.5 upon addition of the dosage unit to 50 mL of 0.1 N HCl(aq).

In certain embodiments, the pH buffering agent may comprise a phosphate, an alginate, or a combination thereof. In certain embodiments, the pH buffering agent may comprise glycerol phosphate, monocalcium phosphate, dicalcium phosphate, tricalcium phosphate, or a combination thereof.

In certain embodiments, the pH buffering agent and the tonicity buffering agent may be present in a ratio by weight of about 1:100 to about 100:1. In certain embodiments, the pH buffering agent and the tonicity buffering agent may be present in a ratio by weight of at least about 1:100, at least about 1:90, at least about 1:80, at least about 1:70, at least about 1:60, at least about 1:50, at least about 1:40, at least about 1:30, at least about 1:20, at least about 1:15, at least about 1:10, at least about 1:9, at least about 1:8, at least about 1:7, at least about 1:6, at least about 1:5, at least about 1:4, at least about 1:3, at least about 1:2, at least about 1:1, at least about 2:1, at least about 3:1, at least about 4:1, at least about 5:1, at least about 6:1, at least about 7:1, at least about 8:1, at least about 9:1, at least about 10:1, at least about 15:1, at least about 20:1, at least about 25:1, at least about 30:1, at least about 40:1, at least about 50:1, at least about 60:1, at least about 70:1, at least about 80:1, at least about 90:1, or at least about 100:1. In certain embodiments, the pH buffering agent and the tonicity buffering agent may be present in a ratio by weight of at most about 100:1, at most about 90:1, at most about 80:1, at most about 70:1, at most about 60:1, at most about 50:1, at most about 40:1, at most about 30:1, at most about 25:1, at most about 20:1, at most about 15:1, at most about 10:1, at most about 9:1, at most about 8:1, at most about 7:1, at most about 6:1, at most about 5:1, at most about 4:1, at most about 3:1, at most about 2:1, at most about 1:1, at most about 1:2, at most about 1:3, at most about 1:4, at most about 1:5, at most about 1:6, at most about 1:7, at most about 1:8, at most about 1:9, at most about 1:10, at most about 1:15, at most about 1:20, at most about 1:25, at most about 1:30, at most about 1:35, at most about 1:40, at most about 1:50, at most about 1:60, at most about 1:70, at most about 1:80, at most about 1:90, or at most about 1:100.

In certain embodiments, the polymeric matrix component may be present in an amount of about 0.1% to about 60% by weight of the dosage unit. In certain embodiments, the polymeric matrix component may be present in an amount of at least about 0.1%, at least about 1.0%, at least about 2.0%, at least about 3.0%, at least about 4.0%, at least about 5.0%, at least about 6.0%, at least about 7.0%, at least about 8.0%, at least about 9.0%, at least about 10.0%, at least about 11.0%, at least about 12.0%, at least about 13.0%, at least about 14.0%, at least about 15.0%, at least about 20.0%, at least about 25.0%, at least about 30.0%, at least about 35.0%, at least about 40.0%, at least about 45.0%, at least about 50.0%, at least about 55.0%, at least about 60.0%, at least about 65.0%, at least about 70.0%, or at least about 75.0% by weight of the dosage unit. In certain embodiments, the polymeric matrix component may be present in an amount of at most about 75.0%, at most about 70.0%, at most about 65.0%, at most about 60.0%, at most about 55.0%, at most about 50.0%, at most about 45.0%, at most about 40.0%, at most about 35.0%, at most about 30.0%, at most about 25.0%, at most about 20.0%, at most about 15.0%, at most about 14.0%, at most about 13.0%, at most about 12.0%, at most about 11.0%, at most about 10.0%, at most about 9.0%, at most about 8.0%, at most about 7.0%, at most about 6.0%, at most about 5.0%, at most about 4.0%, at most about 3.0%, at most about 2.0%, or at most about 1.0% by weight of the dosage unit. In certain embodiments, the polymeric matrix component may be present in an amount of about 50 mg to about 5 g.

In certain embodiments, the polymeric matrix component may comprise an alginate, a carrageenan, a xanthan gum, or a combination thereof.

In certain embodiments, the polymeric matrix component and the tonicity buffering agent may be present in a ratio by weight of about 1:100 to about 100:1. In certain embodiments, the polymeric matrix component and the tonicity buffering agent may be present in a ratio by weight of at least about 1:100, at least about 1:90, at least about 1:80, at least about 1:70, at least about 1:60, at least about 1:50, at least about 1:40, at least about 1:30, at least about 1:20, at least about 1:15, at least about 1:10, at least about 1:9, at least about 1:8, at least about 1:7, at least about 1:6, at least about 1:5, at least about 1:4, at least about 1:3, at least about 1:2, at least about 1:1, at least about 2:1, at least about 3:1, at least about 4:1, at least about 5:1, at least about 6:1, at least about 7:1, at least about 8:1, at least about 9:1, at least about 10:1, at least about 15:1, at least about 20:1, at least about 25:1, at least about 30:1, at least about 40:1, at least about 50:1, at least about 60:1, at least about 70:1, at least about 80:1, at least about 90:1, or at least about 100:1. In certain embodiments, the polymeric matrix component and the tonicity buffering agent may be present in a ratio by weight of at most about 100:1, at most about 90:1, at most about 80:1, at most about 70:1, at most about 60:1, at most about 50:1, at most about 40:1, at most about 30:1, at most about 25:1, at most about 20:1, at most about 15:1, at most about 10:1, at most about 9:1, at most about 8:1, at most about 7:1, at most about 6:1, at most about 5:1, at most about 4:1, at most about 3:1, at most about 2:1, at most about 1:1, at most about 1:2, at most about 1:3, at most about 1:4, at most about 1:5, at most about 1:6, at most about 1:7, at most about 1:8, at most about 1:9, at most about 1:10, at most about 1:15, at most about 1:20, at most about 1:25, at most about 1:30, at most about 1:35, at most about 1:40, at most about 1:50, at most about 1:60, at most about 1:70, at most about 1:80, at most about 1:90, or at most about 1:100.

The viability of the probiotic component of the present invention can be assessed by a suitable method known to a person having ordinary skill in the relevant art. Examples of suitable methods for assessing viability of the probiotic component of the present invention include, but are not limited to, microbiological count methods disclosed in Standard Methods for the Examination of Dairy Products, 16^{th} Edition, 1992, Chapter 6, pages 213-246; and International Dairy Federation Method number 117A: 1988, as recommended by the National Nutritional Food Association (September 1992) (“Probiotic Enumeration Method”).

In certain embodiments, at least about 10% of the probiotic component may remain viable after about 1 week of storage at −10 ° C. and less than 0.1 water activity as measured by Probiotic Enumeration Method. In certain embodiments, at least about 0.1%, at least about 0.5%, at least about 1.0%, at least about 5.0%, at least about 10.0%, at least about 15.0%, at least about 20.0%, at least about 25.0%, at least about 30.0%, at least about 35.0%, at least about 40.0%, at least about 45.0%, at least about 50.0%, at least about 60.0%, at least about 70.0%, at least about 80.0%, or at least about 90.0% of the probiotic component may remain viable as measured by Probiotic Enumeration Method. In certain embodiments, the probiotic component may remain viable after at least about 1 minute, at least about 10 minutes, at least about 30 minutes, at least about 1 hour, at least about 2 hours, at least about 4 hours, at least about 12 hours, at least about 1 day, at least about 2 days, at least about 3 days, at least about 1 week, at least about 2 weeks, at least about 3 weeks, at least about 1 month, at least about 2 months, at least about 3 months, at least about 6 months, at least about 9 months, or at least about 1 year of storage at −10 ° C. and less than 0.1 water activity.

In certain embodiments, at least about 10% of the probiotic component may remain viable at least about 5 minutes after adding the dosage unit to 50 mL of distilled water at 20 ° C. as measured by Probiotic Enumeration Method. In certain embodiments, at least about 0.1%, at least about 0.5%, at least about 1.0%, at least about 5.0%, at least about 10.0%, at least about 15.0%, at least about 20.0%, at least about 25.0%, at least about 30.0%, at least about 35.0%, at least about 40.0%, at least about 45.0%, at least about 50.0%, at least about 60.0%, at least about 70.0%, at least about 80.0%, or at least about 90.0% of the probiotic component may remain viable as measured by Probiotic Enumeration Method. In certain embodiments, the probiotic component may remain viable at least about 5 seconds, at least about 10 seconds, at least about 20 seconds, at least about 30 seconds, at least about 45 seconds, at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 15 minutes, at least about 20 minutes, at least about 25 minutes, at least about 30 minutes, at least about 1 hour, at least about 2 hours, at least about 4 hours, at least about 12 hours, at least about 1 day, at least about 2 days, at least about 3 days, at least about 1 week, at least about 2 weeks, at least about 3 weeks, or at least about 1 month after adding the dosage unit to 50 mL of distilled water at 20 ° C.

In certain embodiments, at least about 10% of the probiotic component may remain viable at least about 5 minutes after adding the dosage unit to 50 mL of 0.1 N HCl(aq) at 20 ° C. as measured by Probiotic Enumeration Method. In certain embodiments, at least about 0.1%, at least about 0.5%, at least about 1.0%, at least about 5.0%, at least about 10.0%, at least about 15.0%, at least about 20.0%, at least about 25.0%, at least about 30.0%, at least about 35.0%, at least about 40.0%, at least about 45.0%, at least about 50.0%, at least about 60.0%, at least about 70.0%, at least about 80.0%, or at least about 90.0% of the probiotic component may remain viable as measured by Probiotic Enumeration Method. In certain embodiments, the probiotic component may remain viable at least about 5 seconds, at least about 10 seconds, at least about 20 seconds, at least about 30 seconds, at least about 45 seconds, at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 15 minutes, at least about 20 minutes, at least about 25 minutes, at least about 30 minutes, at least about 1 hour, at least about 2 hours, at least about 4 hours, at least about 12 hours, at least about 1 day, at least about 2 days, at least about 3 days, at least about 1 week, at least about 2 weeks, at least about 3 weeks, or at least about 1 month after adding the dosage unit to 50 mL of 0.1 N HCl at 20 ° C.

In certain embodiments, the dosage unit may be a particulate, a capsule, a tablet, an ampoule, a suppository, a softgel, a sol-gel, a pill, a powder, a sachet, a blast-cap, or a combination thereof.

This disclosure also provides kits. In certain embodiments, the kit may comprise a dosage unit as described herein and instructions for use.

In certain embodiments, the instructions for use may identify one or more suitable volume and type of liquid to which the dosage unit can be added while retaining viability of at least about 10% of the probiotic component after at least about 5 minutes as measured by Probiotic Enumeration Method. In certain embodiments, the instructions may comprise a suitable temperature at which the dosage unit can be added to the suitable volume and type of liquid while retaining viability of at least about 10% of the probiotic component after at least about 5 minutes as measured by Probiotic Enumeration Method. Volumes and types of liquids and temperatures disclosed elsewhere in this disclosure are suitable for use in the instructions.

This disclosure also provides uses of the dosage units described herein. In certain embodiments, the dosage units described herein may be used for oral or anal delivery of the probiotic component to a portion of the digestive tract of a mammal.

In certain embodiments, the portion of the digestive tract comprises one or more of the mouth cavity, the esophagus, the stomach, the duodenum, the jejunum, the ileum, and the colon.

In certain embodiments, the mammal may be an aardvark, an armadillo, a sloth, an anteater, a bat, a carnivore, a cetacean, a colugo, a shrew, an elephant, a hare, a rabbit, a pika, an even-toed hoofed mammal, an odd-toed hoofed mammal, a hyrax, an insectivore, a marsupial, a pangolin, a primate, a rodent, a seal, a sea lion, or a sirenian. In certain embodiments, the mammal may be a primate. In certain embodiments, the primate may be a monkey, an ape, or a homo sapien.

What is claimed is:

1. A dosage unit comprising a probiotic component, a pH buffering agent, and a tonicity buffering agent.

2. The dosage unit of claim 1, wherein the pH buffering agent is present in an amount suitable to provide a pH of about 1 to about 7 upon addition of the dosage unit to 50 mL of 0.1 N HCl (aq).

3. The dosage unit of claim 1, wherein the pH buffering agent comprises a phosphate, an alginate, or a combination thereof.

4. The dosage unit of claim 1, wherein the pH buffering agent comprises glycerol phosphate, monocalcium phosphate, dicalcium phosphate, tricalcium phosphate, or a combination thereof.

5. The dosage unit of claim 1, wherein the probiotic component comprises lactic acid bacteria, yeast, propionic acid bacteria, or a combination thereof.

6. The dosage unit of claim 1, wherein the probiotic component comprises one or more of Lactobacillus acidophilus, Lactobacillus brevis, Lactobacillus casei, Lactobacillus delbrueckii, Lactobacillus gasseri, Lactobacillus fermentum, Lactobacillus helveticus, Lactobacillus paracasei, Lactobacillus plantarum, Lactobacillus reuteri, Lactobacillus rhamnosus, Lactobacillus salivarius, Lactobacillus lactis, Bifidobacterium bifidum, Bifidobacterium breve, Bifidobacterium infantis, Bifidobacterium lactis, Bifidobacterium longum, Streptococcus thermophiles, and Saccaromyces boulardii.

7. The dosage unit of claim 1, wherein the dosage unit comprises a spore, a Bacillales bacterium, or a combination thereof in an amount of less than about 0.1% by weight of the dosage unit.

8. The dosage unit of claim 1, wherein the tonicity buffering agent is present in an amount suitable to provide an isotonic medium upon addition of the dosage unit to 50 mL of distilled water at 20 ° C.

9. The dosage unit of claim 1, wherein the tonicity buffering agent comprises sodium chloride, potassium chloride, magnesium chloride, calcium chloride, sodium fluoride, potassium fluoride, magnesium fluoride, calcium fluoride, sodium bromide, potassium bromide, magnesium bromide, calcium bromide, sodium iodide, potassium iodide, magnesium iodide, calcium iodide, sodium sulfate, potassium sulfate, magnesium sulfate, calcium sulfate, or a combination thereof.

10. The dosage unit of claim 1, wherein at least about 10% of the probiotic component remains viable after about 1 week of storage at −10 ° C. and less than 0.1 water activity as measured by Probiotic Enumeration Method.

11. The dosage unit of claim 1, wherein at least about 10% of the probiotic component remains viable at least about 5 minutes after adding the dosage unit to 50 mL of distilled water at 20 ° C. as measured by Probiotic Enumeration Method, wherein at least about 10% of the probiotic component remains viable at least about 5 minutes after adding the dosage unit to 50 mL of 0.1 N HCl (aq) at 20 ° C. as measured by Probiotic Enumeration Method, or a combination thereof.

12. A kit comprising the dosage unit of claim 1 and instructions for use.

13. A method of using the dosage unit of claim 1, comprising orally or anally delivering the probiotic component to a portion of the digestive tract of a mammal.

14. A dosage unit comprising a probiotic component, a polymeric matrix component, and a tonicity buffering agent, wherein the tonicity buffering agent is present in an amount and configuration suitable to provide a localized isotonic medium upon addition of the dosage unit to 50 mL of 0.1 N HCl (aq).

15. The dosage unit of claim 14, wherein the polymeric matrix component is present in an amount of about 0.1% to about 60% by weight of the dosage unit.

16. The dosage unit of claim 14, wherein the polymeric matrix component comprises an alginate, a carrageenan, a xanthan gum, or a combination thereof.

17. The dosage unit of claim 14, wherein the probiotic component comprises lactic acid bacteria, yeast, propionic acid bacteria, or a combination thereof.

18. The dosage unit of claim 14, wherein the probiotic component comprises one or more of Lactobacillus acidophilus, Lactobacillus brevis, Lactobacillus casei, Lactobacillus delbrueckii, Lactobacillus gasseri, Lactobacillus fermentum, Lactobacillus helveticus, Lactobacillus paracasei, Lactobacillus plantarum, Lactobacillus reuteri, Lactobacillus rhamnosus, Lactobacillus salivarius, Lactobacillus lactis, Bifidobacterium bifidum, Bifidobacterium breve, Bifidobacterium infantis, Bifidobacterium lactis, Bifidobacterium longum, Streptococcus thermophiles, and Saccaromyces boulardii.

19. The dosage unit of claim 14, wherein the dosage unit comprises a spore, a Bacillales bacterium, or a combination thereof in an amount of less than about 0.1% by weight of the dosage unit.

20. The dosage unit of claim 14, wherein the tonicity buffering agent is present in an amount suitable to provide an isotonic medium upon addition of the dosage unit to 50 mL of distilled water at 20 ° C.

21. The dosage unit of claim 14, wherein the tonicity buffering agent comprises sodium chloride, potassium chloride, magnesium chloride, calcium chloride, sodium fluoride, potassium fluoride, magnesium fluoride, calcium fluoride, sodium bromide, potassium bromide, magnesium bromide, calcium bromide, sodium iodide, potassium iodide, magnesium iodide, calcium iodide, sodium sulfate, potassium sulfate, magnesium sulfate, calcium sulfate, or a combination thereof.

22. The dosage unit of claim 14, wherein at least about 10% of the probiotic component remains viable after about 1 week of storage at −10 ° C. and less than 0.1 water activity as measured by Probiotic Enumeration Method.

23. The dosage unit of claim 14, wherein at least about 10% of the probiotic component remains viable at least about 5 minutes after adding the dosage unit to 50 mL of distilled water at 20 ° C. as measured by Probiotic Enumeration Method, wherein at least about 10% of the probiotic component remains viable at least about 5 minutes after adding the dosage unit to 50 mL of 0.1 N HCl (aq) at 20 ° C. as measured by Probiotic Enumeration Method, or a combination thereof.

24. A kit comprising the dosage unit of claim 14 and instructions for use.

25. A method of using the dosage unit of claim 14, comprising orally or anally delivering the probiotic component to a portion of the digestive tract of a mammal.
